(19) United States
(12) Patent Application Publication
(10) Pub. No.: US 2018/0207103 A1
(43) Pub. Date: Jul. 26, 2018

(54) USE OF AN INJECTABLE ANTIMICROBIAL COMPOSITION FOR THE PREVENTION AND/OR TREATMENT OF OSTEOARTHRITIS

(71) Applicant: CorMedix Inc., Berkeley Heights, NJ (US)

(72) Inventors: Bruce E. Reidenberg, Rye, NY (US); Robert DiLuccio, Haymarket, VA (US)

(21) Appl. No.: 15/928,988

(22) Filed: Mar. 22, 2018

Related U.S. Application Data
(63) Continuation-in-part of application No. 15/252,990, filed on Aug. 31, 2016.
Application No. 15/252,990 is a continuation-in-part of application No. 15/287,822, filed on Oct. 7, 2016.
Application No. 15/287,822 is a continuation-in-part of application No. 15/861,248, filed on Jan. 3, 2018.
Application No. 15/861,248 is a continuation-in-part of application No. 15/858,228, filed on Dec. 29, 2017.
Application No. 15/858,228 is a continuation-in-part of application No. 15/287,822, filed on Oct. 7, 2016.
(60) Provisional application No. 62/211,922, filed on Aug. 31, 2015.
(60) Provisional application No. 62/211,904, filed on Aug. 31, 2015.
(60) Provisional application No. 62/238,167, filed on Oct. 7, 2015.
(60) Provisional application No. 62/442,778, filed on Jan. 5, 2017.
(60) Provisional application No. 62/238,167, filed on Oct. 7, 2015.
(60) Provisional application No. 62/440,054, filed on Dec. 29, 2016.
(60) Provisional application No. 62/474,695, filed on Mar. 22, 2017.

(51) Int. Cl. A61K 9/51; A61K 31/549; A61K 9/10; A61K 9/00; A61P 19/02; A61P 31/00

(52) U.S. Cl. CPC A61K 9/5123; A61K 31/549; A61P 31/00; A61K 9/0019; A61P 19/02; A61K 9/10

(57) ABSTRACT

In one form of the invention, there is provided a method for treating osteoarthritis, the method comprising applying a broad spectrum antimicrobial formulation to the subchondral bone of a mammal. In another form of the invention, there is provided a pharmaceutical composition for treating infections, including infections leading to arthritis.

### REFERENCE TO PENDING PRIOR PATENT APPLICATIONS

This patent application:

(i) is a continuation-in-part of pending prior U.S. patent application Ser. No. 15/252,990, filed Aug. 31, 2016 by CorMedix Inc. and Robert DiLuccio et al. for COMPOSITIONS FOR THE TREATMENT OF JOINTS (Attorney's Docket No. CORMEDIX-0812), which patent application:
(a) claims benefit of prior U.S. Provisional Patent Application Ser. No. 62/211,922, filed Aug. 31, 2015 by CorMedix Inc. and Robert DiLuccio et al. for ANTIMICROBIAL COMPOSITIONS FOR TREATMENT OF JOINTS (Attorney's Docket No. CORMEDIX-8 PROV); and
(b) claims benefit of prior U.S. Provisional Patent Application Ser. No. 62/211,904, filed Aug. 31, 2015 by CorMedix Inc. and Robert DiLuccio et al. for INTRA-ARTICULAR FORMULATION OF TAUROLIDINE (Attorney's Docket No. CORMEDIX-12 PROV);

(ii) is a continuation-in-part of pending prior U.S. patent application Ser. No. 15/287,822, filed Oct. 7, 2016 by CorMedix Inc. and Bruce Reidenberg et al. for SKIN-PENETRATING FORMULATION OF TAUROLIDINE (Attorney's Docket No. CORMEDIX-13), which patent application:
(a) claims benefit of prior U.S. Provisional Patent Application Ser. No. 62/238,167, filed Oct. 7, 2015 by CorMedix Inc. and Bruce Reidenberg et al. for SKIN-PENETRATING FORMULATION OF TAUROLIDINE (Attorney's Docket No. CORMEDIX-13 PROV);

(iii) is a continuation-in-part of pending prior U.S. patent application Ser. No. 15/861,248, filed Jan. 3, 2018 by CorMedix Inc. and Robert DiLuccio for ANTIMICROBIAL DELIVERY SYSTEM FOR THE PREVENTION AND TREATMENT OF INFECTIONS IN THE COLON (Attorney's Docket No. CORMEDIX-19), which patent application:
(a) claims benefit of prior U.S. Provisional Patent Application Ser. No. 62/442,778, filed Jan. 5, 2017 by Cormedix, Inc. and Robert DiLuccio for ANTIMICROBIAL DELIVERY SYSTEM FOR THE PREVENTION AND TREATMENT OF INFECTIONS IN THE COLON (Attorney's Docket No. CORMEDIX-19 PROV);

(iv) is a continuation-in-part of pending prior U.S. patent application Ser. No. 15/858,228, filed Dec. 29, 2017 by CorMedix Inc. and Bruce Reidenberg et al. for SKIN-PENETRATING FORMULATION OF TAUROLIDINE (Attorney's Docket No. CORMEDIX-20), which patent application:
(a) is a continuation-in-part of pending prior U.S. patent application Ser. No. 15/287,822, filed Oct. 7, 2016 by CorMedix Inc. and Bruce Reidenberg et al. for SKIN-PENETRATING FORMULATION OF TAUROLIDINE (Attorney's Docket No. CORMEDIX-13), which patent application in turn claims benefit of:
   (i) prior U.S. Provisional Patent Application Ser. No. 62/238,167, filed Oct. 7, 2015 by CorMedix Inc. and Bruce Reidenberg et al. for SKIN-PENETRATING FORMULATION OF TAUROLIDINE (Attorney's Docket No. CORMEDIX-13 PROV); and
(b) claims benefit of prior U.S. Provisional Patent Application Ser. No. 62/440,054, filed Dec. 29, 2016 by CorMedix Inc. and Bruce Reidenberg et al. for SKIN-PENETRATING FORMULATION OF TAUROLIDINE (Attorney's Docket No. CORMEDIX-20 PROV); and

(v) claims benefit of pending prior U.S. Provisional Patent Application Ser. No. 62/474,695, filed Mar. 22, 2017 by CorMedix Inc. and Bruce E. Reidenberg et al. for USE OF INJECTABLE ANTIMICROBIAL FOR THE PREVENTION AND/OR TREATMENT OF OSTEOARTHRITIS (Attorney's Docket No. CORMEDIX-17 PROV).

The ten (10) above-identified patent applications are hereby incorporated herein by reference.

### FIELD OF THE INVENTION

This invention relates to therapeutic compositions in general, and more particularly to therapeutic compositions for the prevention and/or treatment of osteoarthritis.

### BACKGROUND OF THE INVENTION

Osteoarthritis

Osteoarthritis (OA) is the most common cause of physical disability in the U.S., affecting more than 27 million people (American Academy of Orthopedic Surgeons, The Burden of Musculoskeletal Diseases in the United States: Prevalence, Societal and Economic Cost, American Academy of Orthopaedic Surgeons, Rosemont, Ill., 2008; Lawrence R C, Felson D T, Helmick C G, et al., 2008, National Arthritis Data Workgroup: Estimates of the prevalence of arthritis and other rheumatic conditions in the United States: Part II, Arthritis Rheum: 58(1), 26-35; Centers for Disease Control and Prevention (CDC), 2007-2009, Prevalence of doctor-diagnosed arthritis and arthritis-attributable activity limitation—United States, Morb Mortal Wkly Rep 2010: 59(39), 1261-1265; and Cameron K L, Hsiao M S, Owens B D, et al., 2011, Incidence of physician-diagnosed osteoarthritis among active duty United States military service members, Arthritis Rheum: 63(10), 2974-2982). This disease poses a significant economic burden, with estimated annual costs exceeding $60 billion (Elders M J, 2000, The increasing impact of arthritis on Public Health, JRheumatol: 27, 6-8; and Lawrence R C, Felson D T, Helmick C G, et al., 2008, National Arthritis Data Workgroup: Estimates of the prevalence of arthritis and other rheumatic conditions in the United States: Part II, Arthritis Rheum: 58(1), 26-35), and costs are expected to reach almost $100 billion by 2020 (Oliviero F, Ramonda R, Punzi L, 2010, New horizons in osteoarthritis, SwissMedWkly: 140, w13098).

Post-Traumatic Osteoarthritis (PTOA) accounts for 12% of all cases of OA (Academy of Orthopedic Surgeons, The Burden of Musculoskeletal Diseases in the United States: Prevalence, Societal and Economic Cost, American Academy of Orthopaedic Surgeons, Rosemont, Ill., 2008). PTOA is the loss of cartilage in a joint following trauma. This is a separate condition from an infection of a bone and/or joint, since it is due to the inoculation of bacteria into the bone and/or joint due to the trauma itself. Inasmuch as PTOA primarily affects younger individuals (Lawrence R C, Felson D T, Helmick C G, et al., 2008, National Arthritis Data Workgroup: Estimates of the prevalence of arthritis and other rheumatic conditions in the United States: Part II, Arthritis Rheum: 58(1), 26-35; and Centers for Disease Control and Prevention (CDC), 2007-2009, Prevalence of doctor-diagnosed arthritis and arthritis-attributable activity limitation-United States, Morb Mortal Wkly Rep 2010: 59(39), 1261-1265), it leads to reduced physical activity and to deconditioning of the musculoskeletal system. Joint replacement in this young patient group is complicated by the limited lifespan of joint implants.

PTOA has been documented in many joints, but military data shows the highest prevalence of PTOA in the knee (Cameron K L, Hsiao M S, Owens B D, et al., 2011, Incidence of physician-diagnosed osteoarthritis among active duty United States military service members, Arthritis Rheum: 63(10); 2974-2982).

Animal models of PTOA appear to show the primary lesion to be in the subchondral bone (Elders M J, 2000, The increasing impact of arthritis on Public Health, J Rheumatol: 27; 6-8). See FIG. 1, which shows the location of subchondral bone in a knee joint. It appears that the response to trauma includes the release and/or inhibition of a variety of growth factors and cytokines (Oliviero F, Ramonda R, Punzi L, 2010, New horizons in osteoarthritis, Swiss Med Wkly: 140, w13098; and Brown T D, Johnston R C, Saltzman C L, Marsh J L, Buckwalter J A, Posttraumatic osteoarthritis: a first estimate of incidence, prevalence, and burden of disease, J Orthop Trauma, 2006, 20:739-744). It is likely that disruption of the healing process, by an imbalance of chemical signaling, and/or genetic deficiency of signaling, and/or repeated trauma preventing full healing, results in OA and loss of joint function.

To date, no evaluation of chronic infection of the subchondral bone, such as Proprionibacterium acne found in intervertebral discs (Rollason J, McDowell A, Albert H B, Barnard E, Worthington T, Hilton A C, Vernallis A, Patrick S, Elliott T, Lambert P. Genotypic and antimicrobial characterisation of Propionibacterium acnes isolates from surgically excised lumbar disc herniations, Biomed Res Int. 2013 Aug. 28), has been published.

Current Methods for Treating OA

The treatment of OA generally involves a combination of exercise or physical therapy, lifestyle modification, and analgesics.

Two major guidelines have been published for the treatment of osteoarthritis of the knee. The OsteoArthritis Research Society International (OARSI) (2014), and the American Academy of Orthopedic Surgeons (AAOS) (2013), agree that physical therapy and weight loss for the obese are beneficial (T. E. McAlindon, R. R. Bannuru, M. C. Sullivan, N. K. Arden, F. Berenbaum, S. M. Bierma-Zeinstra, G. A. Hawker, Y. Henrotin, D. J. Hunter, H. Kawaguchi, K. Kwoh, S. Lohmander, F. Rannou, E. M. Roos, M. Underwood, OARSI guidelines for the non-surgical management of knee osteoarthritis, Osteoarthritis and Cartilage 22, 2014, 363-388; and TREATMENT OF OSTEOARTHRITIS OF THE KNEE EVIDENCE-BASED GUIDELINE, 2ND EDITION, Adopted by the American Academy of Orthopaedic Surgeons Board of Directors, May 18, 2013, http://www.aaos.org/research/guidelines/TreatmentofOst eoarthritisoftheKneeGuideline.pdf, Downloaded 31 Jul. 2016).

The OARSI and the AAOS also agree that a number of treatments cannot be recommended due to lack of evidence: acupuncture, balneotherapy for the knee, chondroitin, glucosamine, ultrasound and electrotherapy.

Pharmacologically, the OARSI considers acetaminophen and topical capsaicin “appropriate”, in addition to Non-Steroidal Anti-Inflammatory Drugs (NSAIDs). However, the AAOS recommends only NSAIDs and states that there is insufficient data to recommend acetaminophen, opiates or pain patches. Interestingly, the AAOS also found the data for intra-articular steroids inconclusive, while the OARSI found intra-articular steroids to be “appropriate”. The AAOS “cannot recommend” hyaluronic acid injection into osteoarthritic knees and the OARSI states that the data are “uncertain.”

To date, there is no proven treatment to slow or reverse OA. With growing financial pressure on healthcare systems and ever-increasing numbers of patients, there is an urgent need for a new approach for treating and/or preventing OA.

Animal models of PTOA appear to show the primary lesion to be in the subchondral bone (Elsaid K A, Zhang L, Shaman Z, Patel C, Schmidt T A, Jay G D, The impact of early intra-articular administration of interleukin-1 receptor antagonist on lubricin metabolism and cartilage degeneration in an anterior cruciate ligament transection model, Osteoarthritis Cartilage, 2015 Jan., 23(1):114-21, doi: 10.1016/j.joca.2014.09.006, Epub 2014 Sep. 16). It appears that the response to trauma includes the release and/or inhibition of a variety of growth factors and cytokines (Zlotnicki J P, Geeslin A G, Murray I R, Petrigliano F A, LaPrade R F, Mann B J, Musahl V. Biologic Treatments for Sports Injuries II Think Tank-Current Concepts, Future Research, and Barriers to Advancement, Part 3: Articular Cartilage, Orthop J Sports Med, 2016 Apr. 15, 4(4) 2325967116642433; and Lotz MK1, Kraus V B, New developments in osteoarthritis, Posttraumatic osteoarthritis: pathogenesis and pharmacological treatment options, Arthritis Res Ther. 2010, 12(3):211, doi: 10.1186/ar3046, Epub 2010 Jun. 28). It is likely that disruption of the healing process, by an imbalance of chemical signaling, and/or genetic deficiency of signaling, and/or repeated trauma preventing full healing, results in osteoarthritis and loss of joint function. To date, no evaluation of chronic infection of the subchondral bone, such as the Propionibacterium acne found in intervertebral discs (Rollason J, McDowell A, Albert H B, Barnard E, Worthington T, Hilton A C, Vernallis A, Patrick S, Elliott T, Lambert P. Genotypic and antimicrobial characterization of Propionibacterium acnes isolates from surgically excised lumbar disc herniations, Biomed Res Int. 2013 Aug. 28), has been published.

Evidence of Subclinical Infection as Etiology of Herniated Vertebral Discs

Rollason et al. (Rollason J, McDowell A, Albert H B, Barnard E, Worthington T, Hilton A C, Vernallis A, Patrick S, Elliott T, Lambert P. Genotypic and antimicrobial characterisation of Propionibacterium acnes isolates from surgically excised lumbar disc herniations, Biomed Res Int. 2013, 2013:530382, doi: 10.1155/2013/530382, Epub 2013 Aug. 28) examined 5 biopsies, each from 64 patients with herniated vertebral discs, and detected Propionibacterium acnes (P. acnes) and other bacteria by anaerobic culture, followed by biochemical and polymerase chain reaction-based (PCR-based) identification. Many of the identified microbes are not frequently found in the skin and were identified in duplicate biopsies, making intra-operative or laboratory contamination unlikely.

Clinical Data that Microfracture of Subchondral Bone May Contribute to Osteoarthritis

There is new clinical evidence that supports the animal model prediction that subchondral bone, as the site of the primary lesion, is a significant cause of PTOA. Among other things, it has been found that surgical microfracture of subchondral bone is deleterious to patients having anterior cruciate ligament (ACL) repair with a full thickness cartilage lesion (Røtterud JH, Sivertsen E A, Forssblad M, Engebretsen L, Årøen A, Effect on Patient-Reported Outcomes of Debridement or Microfracture of Concomitant Full-Thickness Cartilage Lesions in Anterior Cruciate Ligament-Reconstructed Knees: A Nationwide Cohort Study From Norway and Sweden of 357 Patients With 2-Year Follow-up, Am J Sports Med, 2016 February, 44(2):337-44).

Delivery of Antibiotics to Subchondral Bone

The use of depots to deliver antibiotics to subchondral bone has been attempted with a variety of drugs. The use of antibiotic depots allows for high local concentrations of antibiotic with little systemic absorption.

By way of example but not limitation, antibiotics have been delivered with Poly(methyl methacrylate) (PMMA), a common bone cement. Since PMMA produces heat when it is hardening, the active agents (i.e., the antibiotics) generally have to be heat-stable and in powder form. Tobramycin and Vancomycin are the most commonly used antibiotics for depot delivery with PMMA. Antibiotic release is bi-phasic, with most release occurring during the first hours to days post-implantation, and the remaining elution persisting for weeks and sometimes for years.

Some of the other antibiotics that have been tried with PMMA include Clindamycin (which elutes well but is not available as a pharmaceutical grade powder), Fluoroquinolones (results have not yet been reported), Erythromycin (which is heat-stable but has demonstrated inadequate elution from the PMMA), and Tetracycline, Colistin and Gentamicin (which fail to elute from the cement in clinically meaningful quantities).

Most of the “antibiotic cement” use in the United States has been “off-label” use by individual surgeons, and despite very encouraging results from several studies, approval of antibiotic cement has been slow to occur.

There are also newer types of materials available for local delivery of antibiotics which are resorbable and do not require removal (N. V. Kalore, T. J. Gioe, and J. A. Singh, Diagnosis and management of infected total knee arthroplasty, Open Orthop J 5, 2011, 86-91).

However, many antibiotics are beginning to suffer from antibiotic resistance, which occurs as microorganisms naturally mutate to new forms which are resistant to a given antibiotic.

Taurolidine

Taurolidine is a non-toxic, broad spectrum antibacterial and anti-fungal compound.

Taurolidine has also been demonstrated to prevent biofilm formation (Sodemann, K., Polaschegg, H. D., and Feldmer, B., 2001, Two years' experience with Dialock and CLS (a new antimicrobial lock solution), Blood Purif. 19(2): 251-4; and Shah, C. B., et al., 2002, Antimicrobial activity of a novel catheter lock solution, Antimicrob Agents Chemother., 46(6): 1674-9).

Taurolidine is a synthetic molecule developed as an antibacterial agent in the 1970's (Calabresi P, Goulette F A, and Darnowski J W, Taurolidine: cytotoxic and mechanistic evaluation of a novel antineoplastic agent, Cancer Res., 2001 Sep. 15, 61(18):6816-21). Taurolidine is unstable in biologic fluids (it hydrolyzes in water) and is in equilibrium with formaldehyde, methylene glycol and other compounds as described in Gong et al. (Gong L, Greenberg H E, Perhach J L, Waldman S A, Kraft W K, The pharmacokinetics of taurolidine metabolites in healthy volunteers, J Clin Pharmacol., 2007 June, 47(6):697-703, Epub 2007 Mar. 29).

Taurolidine is commercially available in Europe as a 1.35% solution (Neutrolin®, CorMedix Inc.) for preventing the formation of biofilms in central venous catheters, and in Germany, Austria, Switzerland, Poland, and the Netherlands as a 2% solution (Taurolin®, Geistlich Pharma AG) primarily for intraperitoneal use and within the urinary bladder. Intraperitoneal administration of Taurolidine has been shown to significantly reduce morbidity associated with peritonitis (Sodemann, K., et al., Prevention of sepsis in HD Catheters using an antimicrobial lock, American Society of Nephrology, 2001).

Taurolidine has been given systemically to humans in doses of up to 30 grams per day with no significant adverse outcomes (Taylor, C., et al., A New Haemodialysis Catheter-Locking Agent reduces infections in Haemodialysis Patients, Journal of Renal Care 2008, 34(3): p. 116-120).

Significantly, unlike antibiotics, there is no evidence of microorganisms developing a resistance to Taurolidine to date.

### SUMMARY OF THE INVENTION

Osteoarthritis (OA) is the most common cause of disability in the U.S.

Post-Traumatic osteoarthritis (PTOA) accounts for 12% of osteoarthritis cases in the United States and may be due to subclinical infection in the subchondral bone.

The present invention provides for broad spectrum antimicrobial treatment, applied locally to the subchondral bone, to prevent or treat osteoarthritis by limiting cartilage loss from changes in subchondral bone due to infection. The antimicrobial is preferably delivered as a depot to the area of the subchondral bone and the antimicrobial is preferably released over an extended period of time.

The preferred broad spectrum antimicrobial is Taurolidine.

The preferred formulations for subchondral bone injections are injectable gel formulations, nanoparticle formulations or crystal suspension (salt) formulations that provide sustained release of the antimicrobial (e.g., Taurolidine).

In other words, the present invention comprises the provision and use of a broad spectrum (active against many different microorganisms) antimicrobial, applied locally, to treat subclinical infections in the subchondral bone to preserve cartilage in the adjacent joint. The antimicrobial is applied by local injection in the form of a gel formulation, nanoparticle formulation or crystal suspension (salt) formulation that slowly releases the active moiety (methylol groups in the case of Taurolidine) into the subchondral bone. Such localized delivery of the antimicrobial, combined with the delayed release of the antimicrobial, provides effective treatment of the infection, thereby preserving cartilage and joint function.

In one preferred form of the invention, there is provided a method for treating osteoarthritis, the method comprising applying a broad spectrum antimicrobial formulation to the subchondral bone of a mammal.

In another preferred form of the invention, there is provided a pharmaceutical composition for treating infections, the pharmaceutical composition comprising Taurolidine carried by one from the group consisting of: hydrogels, liquids, thixotropic gels, colloidal mixtures, dispersal suspensions, and injectable polymers.

In another preferred form of the invention, there is provided a pharmaceutical composition comprising Taurolidine and at least one selected from the group consisting of a Pluronic formulation; Hyaluronic acid (HA) and water; chitin and water; chitosan (or alginate) and water; and cyclodextrin and water.

In another preferred form of the invention, there is provided a pharmaceutical composition comprising Taurolidine and a polyethylene glycol (PEG)-based hydrogel system.

In another preferred form of the invention, there is provided a pharmaceutical composition comprising Taurolidine and a polyvinylpyrrolidone (PVP)-based hydrogel system.

In another preferred form of the invention, there is provided a pharmaceutical composition comprising Taurolidine in a crystalline salt form suspended in a carrier for administration to subchondral bone.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing the location of subchondral bone in a knee joint;

FIG. 2 is a schematic view showing a nanoparticle comprising a Taurolidine core surrounded by an encapsulant, wherein the encapsulant breaks down over time when exposed to body fluid so as to release the Taurolidine core for hydrolization;

FIG. 3 is a schematic view showing an exemplary time-release profile for the Taurolidine in the nanoparticle shown in FIG. 2;

FIG. 4 is a schematic view showing a nanoparticle comprising Taurolidine core surrounded by an encapsulant, wherein the encapsulant also comprises Taurolidine, and further wherein the encapsulant breaks down over time when exposed to body fluid so as to (i) release the Taurolidine contained within the encapsulant for hydrolization as the encapsulant breaks down, and (ii) release the Taurolidine core for hydrolization after the encapsulant has broken down;

FIG. 5 is a schematic view showing an exemplary time-release profile for the Taurolidine in the nanoparticle shown in FIG. 4;

FIG. 6 is a schematic view showing a nanoparticle omitting the Taurolidine core and formed entirely out of the “encapsulant” material, wherein the encapsulant material comprises Taurolidine dispersed within the encapsulant material, and further wherein the encapsulant material breaks down over time when exposed to body fluid so as to release the Taurolidine contained within the encapsulant material for hydrolization as the encapsulant material breaks down;

FIG. 7 is a schematic view showing an exemplary time-release profile for the Taurolidine in the nanoparticle shown in FIG. 6; and

FIG. 8 is a schematic view showing the antimicrobial composition of the present invention being injected into subchondral bone.

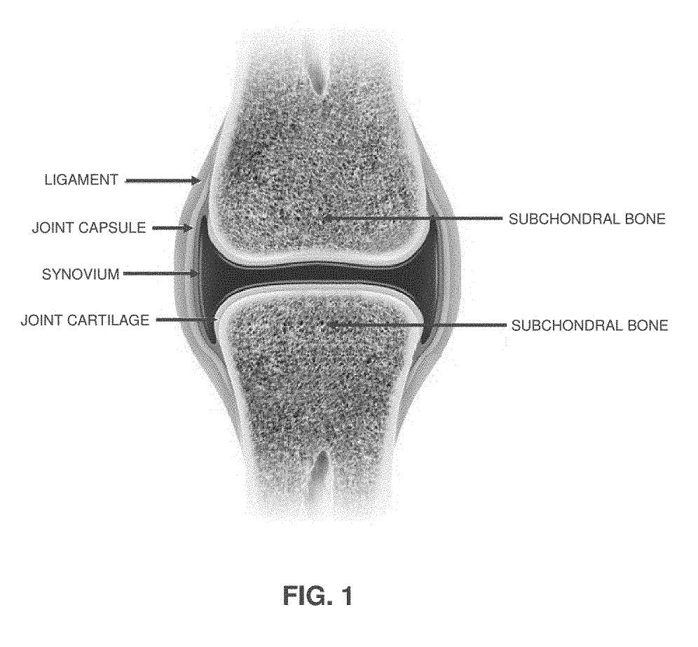

### DETAILED DESCRIPTION OF THE PREFERRED EMBODIMENTS

In one preferred form of the invention, the invention comprises the provision and use of a novel formulation of an antimicrobial designed to deliver the antimicrobial to the subchondral bone, whereby to treat a subclinical infection in the subchondral bone and thus preserve cartilage in adjacent joints, e.g., such as a patient suffering from chronic infections in subchondral bone due to osteoarthritis (OA), including post-traumatic osteoarthritis (PTOA).

More particularly, recent clinical studies show a relationship between subclinical infections in bone and arthritis. Thus, there is now provided a novel method for preventing and/or treating osteoarthritis, wherein the method comprises the delivery of an antimicrobial composition to the subchondral bone, wherein the antimicrobial composition is specifically designed to provide a predictable and therapeutically significant rate of release of the antimicrobial to a localized point of application, i.e., the site of infection within the subchondral bone. The antimicrobial is preferably injected directly into the subchondral bone, e.g., using a syringe. Alternatively, the antimicrobial may be injected into the intramedullary canal of the bone, or into another portion of the bone, such that the antimicrobial migrates into the subchondral bone.

The present invention preferably uses the antimicrobial Taurolidine, which is highly effective against infection. However, Taurolidine is unstable in biologic fluids, inasmuch as the Taurolidine hydrolyzes in water. Therefore, in order to protect the Taurolidine from premature hydrolysis, as well as to provide for the delayed release of the Taurolidine over time, the Taurolidine may be encapsulated (e.g., contained within a nanoparticle) which is carried to the infection site by a suitable vehicle (e.g., a hydrogel, a liquid, a colloidal mixture, etc.). As will hereinafter be discussed in further detail, encapsulating the Taurolidine in a nanoparticle protects the Taurolidine from premature hydrolysis and provides for the delayed release of the Taurolidine over time. As will also hereinafter be discussed in further detail, the delivery vehicle carrying the nanoparticle may also protect the Taurolidine from premature hydrolysis and provide for the delayed release of the Taurolidine over time. Alternatively, the Taurolidine may be delivered to the infection site in another suitable form (e.g., such as a salt suspended in a gel, or as a salt in solution). In such an alternative delivery scheme, the component carrying the Taurolidine (e.g., the gel or solution) may be configured to protect the Taurolidine from premature hydrolysis and provide for the delayed release of the Taurolidine over time.

Novel Pharmaceutical Composition Comprising

Taurolidine Nanoparticles in a Suitable Carrier

In one preferred form of the invention, there is provided a novel pharmaceutical composition which comprises (i) a nanoparticle containing a therapeutically-effective quantity of Taurolidine (e.g., in the form of a saturated solution of Taurolidine, Taurolidine in crystalline form, Taurolidine in combination with another substance, etc.) surrounded by an encapsulant, and (ii) a suitable carrier (e.g., a hydrogel) for carrying the nanoparticle. The encapsulant of the nanoparticle protects the Taurolidine core of the nanoparticle from hydrolysis until the Taurolidine is in the subchondral bone, whereupon the encapsulant breaks down so as to release the Taurolidine core at the site of the infection, with the Taurolidine core then hydrolyzing to its active moieties (i.e., methylol groups), whereby to treat the infection (or to prevent infection). Thus, the nanoparticle comprises a Taurolidine core surrounded by an encapsulant, with the encapsulant protecting the Taurolidine core from premature hydrolization during delivery to the site of the infection, and with the encapsulant naturally breaking down within the body after the nanoparticle has reached the site of the infection, whereby to release the Taurolidine core for hydrolization at the site of the infection.

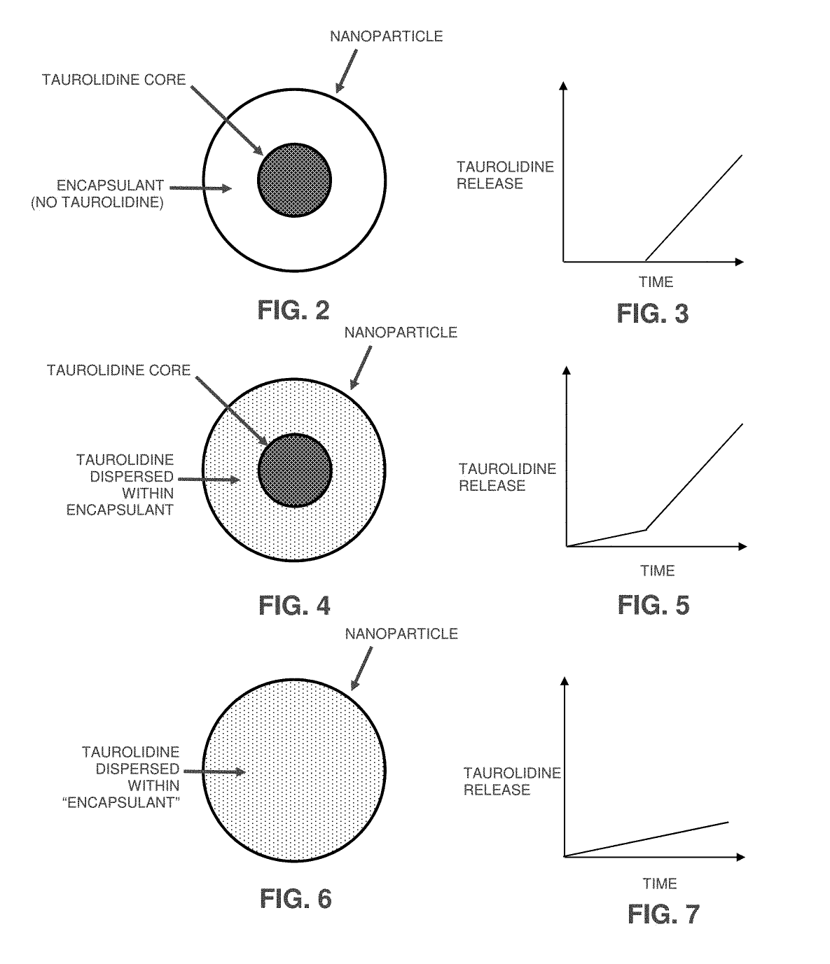

See FIG. 2, which shows a nanoparticle comprising a Taurolidine core surrounded by an encapsulant, wherein the encapsulant breaks down over time when exposed to body fluid so as to release the Taurolidine core for hydrolization. See also FIG. 3, which shows an exemplary time-release profile for the Taurolidine contained in the nanoparticle shown in FIG. 2. It will be appreciated that with the nanoparticle construction shown in FIG. 2, the nanoparticle provides for a delayed release of the Taurolidine. It will also be appreciated that the encapsulant may be specifically engineered so as to provide the desired time-release profile for the Taurolidine core.

In one form of the invention, the nanoparticle comprises a Tauroldine center or core (e.g., in the form of a saturated solution of Taurolidine, Taurolidine in crystalline form, Taurolidine in combination with another substance, etc.) and a glyceride exterior (e.g., mono-, di- or tri-glycerides, or a combination thereof), where the glyceride exterior protects the Taurolidine center from premature hydrolyzation. It will also be appreciated that the glyceride encapsulant may be specifically engineered so as to provide the desired time-release profile for the Taurolidine core.

In another form of the invention, the nanoparticle comprises a Taurolidine center or core (e.g., in the form of a saturated solution of Taurolidine, Taurolidine in crystalline form, Taurolidine in combination with another substance, etc.) and a lipophilic peptide exterior (e.g., saline, leucine, proline, phenylalanine and/or tryptophan), where the lipophilic peptide exterior protects the Taurolidine center from premature hydrolyzation. Again, it will also be appreciated that the lipophilic peptide encapsulant may be specifically engineered so as to provide the desired time-release profile for the Taurolidine core.

The suitable carrier may comprise appropriate hydrogels, liquids, thixotropic gels, colloidal mixtures, dispersal suspensions and/or injectable polymers. Note that the suitable carrier may also protect the Taurolidine from premature hydrolysis while the Tauroldine, and subsequently the nanoparticle, are diffusing through layers of subchondral bone.

If desired, the encapsulant surrounding the Taurolidine core may also comprise Taurolidine, with the Taurolidine being dispersed within the encapsulant. Note that the Taurolidine may be evenly dispersed within the body of the encapsulant, or the Taurolidine may have a concentration gradient within the body of the encapsulant. In the preferred form of the invention, the Taurolidine is evenly dispersed within the body of the encapsulant. See FIG. 4, which shows a nanoparticle comprising a Taurolidine core surrounded by an encapsulant, wherein the encapsulant comprises Taurolidine, and further wherein the encapsulant breaks down over time when exposed to body fluid so as to (i) release the Taurolidine contained within the encapsulant for hydrolization as the encapsulant breaks down, and (ii) release the Taurolidine core for hydrolization after the encapsulant has fully broken down. See also FIG. 5, which shows an exemplary time-release profile for the Taurolidine contained in the nanoparticle shown in FIG. 4. It will be appreciated that with the nanoparticle construction shown in FIG. 4, the nanoparticle provides for a gradual, and then increased, release of the Taurolidine. It will also be appreciated that the encapsulant may be specifically engineered so as to provide the desired time-release profile for the Taurolidine (both the Taurolidine contained in the encapsulant and the Taurolidine contained in the core).

If desired, the nanoparticle may omit the Taurolidine core and be formed entirely out of the “encapsulant” material, wherein the encapsulant material comprises Taurolidine dispersed within the encapsulant material, and further wherein the encapsulant material breaks down over time when exposed to body fluid so as to release the Taurolidine contained within the encapsulant material. Note that in this form of the invention, since the Taurolidine is dispersed within the “encapsulant” material, the encapsulant material is not encapsulating the Taurolidine in the same manner as when the encapsulant material encapsulates a core of Taurolidine, such as described above, however, the term “encapsulant” material may still be used in this form of the invention since the encapsulant material effectively covers the Taurolidine in the nanoparticle. In one preferred form of the invention, the encapsulant material comprises glycerides (e.g., mono-, di-, or tri-glycerides, or a combination thereof). In another preferred form of the invention, the encapsulant material comprises lipophilic peptides (e.g., saline, leucine, proline, phenylalanine and/or tryptophan). In still another form of the invention, the encapsulant material may comprise another material which is consistent with the present invention. Note that the Taurolidine may be evenly dispersed within the body of the encapsulant material, or the Taurolidine may have a concentration gradient within the body of the encapsulant material. In the preferred form of the invention, the Taurolidine is evenly dispersed within the body of the encapsulant material. See FIG. 6, which shows a nanoparticle omitting a Taurolidine core and formed entirely out of the encapsulant material, wherein the encapsulant material comprises Taurolidine dispersed within the encapsulant material, and further wherein the encapsulant material breaks down over time when exposed to body fluid so as to release the Taurolidine contained within the encapsulant material for hydrolization as the encapsulant material breaks down. See also FIG. 7, which shows an exemplary time-release profile for the Taurolidine contained in the nanoparticle shown in FIG. 6. It will be appreciated that with the nanoparticle construction shown in FIG. 6, the nanoparticle provides for a gradual release of the Taurolidine. It will also be appreciated that the encapsulant material may be specifically engineered so as to provide the desired time-release profile for the Taurolidine contained in the encapsulant material.

Taurolidine Contained in a Gel or Solution

In another form of the invention, the novel pharmaceutical composition comprises a therapeutically-effective amount of Taurolidine and a suitable carrier for carrying the Taurolidine to the subchondral bone. In this form of the invention, the suitable carrier may comprise a gel or solution containing the Taurolidine. By way of example but not limitation, the suitable carrier may be Pluronic formulations, hyaluronic acid, chitin and water, chitosan (or alginate) and water, cyclodextrin and water, or a combination thereof.

Taurolidine Contained in a PEG or PVP Hydrogel

In another form of the invention, the novel pharmaceutical composition comprises a therapeutically-effective amount of Taurolidine and a hydrogel for carrying the Taurolidine. In a preferred form of the invention, the hydrogel comprises polyethylene glycol (PEG) or polyvinylpyrrolidone (PVP), and the Taurolidine is carried by the PEG or PVP hydrogel. The PEG or PVP hydrogel provides for delayed and sustained release of the Taurolidine when the pharmaceutical composition is exposed to body fluids.

Taurolidine Salt Suspended in a Suitable Carrier

In yet another form of the invention, the novel pharmaceutical composition comprises a therapeutically-effective amount of Taurolidine in crystallized salt form and a suitable carrier (e.g., a hydrogel where the salt is suspended in the gel, or a solution where the salt is dispersed in the solution).

Method of Use

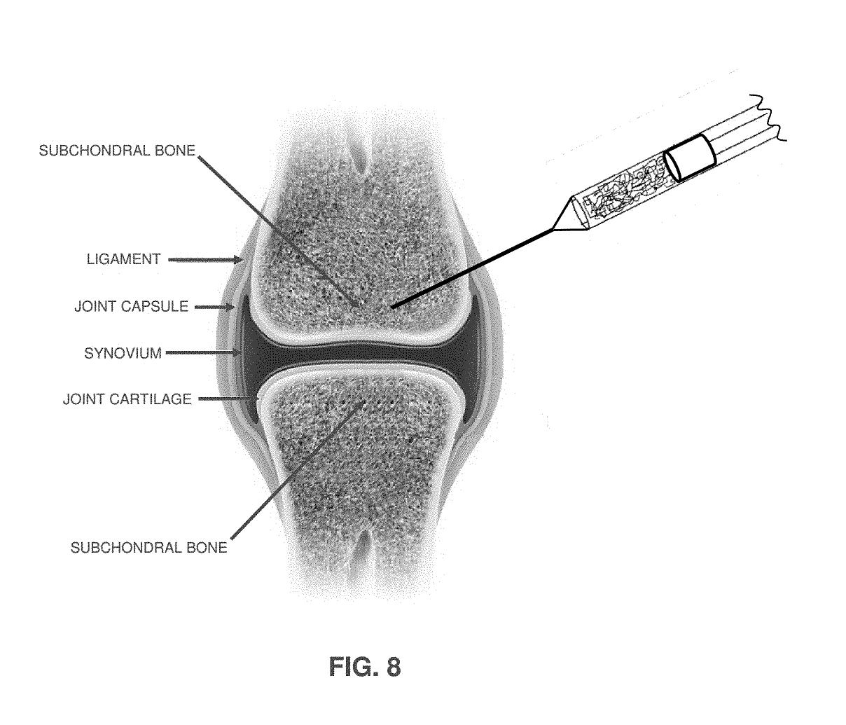

The novel pharmaceutical composition is delivered into the subchondral bone so as to treat or prevent infection in the subchondral bone which could lead to osteoarthritis. In one preferred form of the invention, the novel pharmaceutical composition is injected directly into the subchondral bone using a syringe. See FIG. 8. Alternatively, the pharmaceutical composition may be injected into the intramedullary canal of the bone, or into another portion of the bone, such that the antimicrobial migrates into the subchondral bone.

It should be appreciated that, in the preferred forms of the invention, the hydrolysable Taurolidine is protected from premature hydrolyzation by surrounding the Taurolidine with a “sacrificial” agent, e.g., by encapsulating the hydrolysable Taurolidine within a nanoparticle having a hydrolysable exterior coating, or with the hydrolysable Taurolidine being mixed into a mass of an excipient (e.g., a gel or solution) which shields the hydrolysable Taurolidine from premature exposure to body fluids. When the pharmaceutical composition is applied to the infection site, the “shielded” Taurolidine passes into the subchondral bone without the Taurolidine experiencing substantial hydrolyzation. Once the Taurolidine is within the subchondral bone, the sacrificial agent (e.g., the encapsulant of the nanoparticle or the gel or solution mass of the excipient) breaks down (or otherwise dissipates) and the Taurolidine is released and hydrolyzed, exposing the active moieties (i.e., methylol groups) of the Taurolidine which treat the infection (or prevent infection).

Thus, the present invention comprises the provision and use of a novel pharmaceutical composition which allows for subchondral delivery of therapeutically-effective amounts of Taurolidine to desired regions of bone in order to treat subclinical infections. Furthermore, the present invention provides for shielded delivery of the hydrolysable Taurolidine to the infection site, whereupon the shielding agent (e.g., the shielding encapsulant of a nanoparticle, the shielding mass of a gel or solution) breaks down (or otherwise dissipates) and exposes the Taurolidine for hydrolyzation at the site of the infection.

### Modifications of the Preferred Embodiments

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for treating osteoarthritis, the method comprising applying a broad spectrum antimicrobial formulation to the subchondral bone of a mammal.

2. A method according to claim 1 wherein the broad spectrum antimicrobial formulation is applied to the subchondral bone of a mammal to prevent bone trauma from progressing to osteoarthritis.

3. A method according to claim 1 wherein the broad spectrum antimicrobial formulation is applied to the subchondral bone of a mammal to treat established osteoarthritis.

4. A method according to claim 1 wherein the mammal is a human.

5. A method according to claim 1 wherein the broad spectrum antimicrobial formulation is applied locally into subchondral bone or adjacent to subchondral bone.

6. A method according to claim 1 wherein the broad spectrum antimicrobial formulation inhibits the growth of anaerobic microbes.

7. A method according to claim 1 wherein the broad spectrum antimicrobial formulation comprises Taurolidine.

8. A pharmaceutical composition for treating infections, the pharmaceutical composition comprising Taurolidine carried by one from the group consisting of: hydrogels, liquids, thixotropic gels, colloidal mixtures, dispersal suspensions, and injectable polymers.

9. A pharmaceutical composition according to claim 8 wherein the pharmaceutical composition is configured to provide for a sustained release of Taurolidine at a concentration sufficiently high, and capable of being applied to the region for a sufficient period of time, to treat the infection.

10. A pharmaceutical composition according to claim 9 wherein the pharmaceutical composition is configured to provide for the hydrolysis of Taurolidine to its active methylol moieties in the subchondral bone.

11. A pharmaceutical composition according to claim 8 wherein the Taurolidine is delivered in nanoparticles dispersed in a carrier.

12. A pharmaceutical composition according to claim 8 wherein the nanoparticles comprise a Taurolidine core surrounded by an encapsulant, wherein the encapsulant breaks down when exposed to body fluids.

13. A pharmaceutical composition according to claim 12 wherein the nanoparticles comprise a Taurolidine core encapsulated by a glyceride.

14. A pharmaceutical composition according to claim 13 wherein the glyceride comprises at least one from the group consisting of mono-, di- and tri-glycerides.

15. A pharmaceutical composition according to claim 12 wherein the nanoparticles comprise a Taurolidine core encapsulated by lipophilic peptides.

16. A pharmaceutical composition according to claim 15 wherein the lipophilic peptides comprise at least one from the group consisting of valine, leucine, proline, phenylalanine, tryptophan, and combinations of the foregoing.

17. A pharmaceutical composition according to claim 12 wherein the encapsulant also comprises Taurolidine.

18. A pharmaceutical composition according to claim 12 wherein the nanoparticles comprise a body and Taurolidine dispersed within the body, and further wherein the body breaks down when exposed to body fluids.

19. A pharmaceutical composition according to claim 11 wherein the carrier comprises at least one from the group consisting of hydrogels, liquids, thixotropic gels, colloidal mixtures, dispersal suspensions and injectable polymers.

20. A pharmaceutical composition comprising Taurolidine and at least one selected from the group consisting of a Pluronic formulation; Hyaluronic acid (HA) and water; chitin and water; chitosan (or alginate) and water; and cyclodextrin and water.

21. A pharmaceutical composition comprising Taurolidine and a polyethylene glycol (PEG)-based hydrogel system.

22. A pharmaceutical composition comprising Taurolidine and a polyvinylpyrrolidone (PVP)-based hydrogel system.

23. A pharmaceutical composition comprising Taurolidine in a crystalline salt form suspended in a carrier for administration to subchondral bone.
